(19) United States
(12) Patent Application Publication
(10) Pub. No.: US 2002/0032530 A1
(43) Pub. Date: Mar. 14, 2002

(54) Integrated genomic services

(76) Inventors: Sushma Pati, Woodside, CA (US); Raya Zion, Burlingame, CA (US); Ian Alexander Fairwether Black, Piedmont, CA (US); Christopher Lehman, Sunnyvale, CA (US); Ron Caspi, Mountain View, CA (US); Kathryn M. Stephens, Fremont, CA (US); David Zarling, Menlo Park, CA (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/782,171

(22) Filed: Feb. 12, 2001

Related U.S. Application Data
(60) Provisional application No. 60/182,031, filed on Feb. 11, 2000.

(51) Int. Cl. G06F 17/60; G06F 19/00; G01N 33/48; G01N 33/50

(52) U.S. Cl. 702/20; 705/2

(57) ABSTRACT

The present invention is directed to methods and systems for integrating the provision of genomics services and the production of genomics products. In one aspect of the invention, a method is provided for integrated genomic services comprising (a) receiving a first request from a customer, wherein said request comprises a first nucleic acid sequence, and an order for at least two genomics products or services; and (b) utilizing said nucleic acid sequence to provide said at least two genomics services or products.

This application claims priority to U.S. Provisional Application No. 60/182,031, filed Feb. 11, 2000, which is expressly incorporated herein in its entirety by reference.

### FIELD OF THE INVENTION

The present invention is directed towards integrating the ordering and provision of functional genomic services and products.

### BACKGROUND OF THE INVENTION

Conventional approaches to genotypic and phenotypic screening for biovalidation of targets for pharmaceutical development are hampered by functional genomic processes and/or services that are inherently slow, inefficient, labor intensive, and/or low throughput. The limitations are encountered at every step of the process from gene cloning, target identification, phenotypic screening, small molecule bioassays, drug biovalidation in cells/animals, phenotypic biovalidation in cells/animals, not to mention locating prepackaged kits or service providers and efficiently managing the process to effectively use resources.

In a typical scenario, an expressed sequence tag (EST) or other nucleic acid sequence of interest is used to obtain a clone of the gene or cDNA containing the EST. The EST can be used to search a database such as GeneBank or other proprietary database to obtain partial or full length gene or cDNA sequences within full length gene sequences that match or partially match the given EST. If identical match is found, it is likely that the EST came from the complete gene or possibly from a very highly conserved region of a gene in the same family as the gene from which the EST sequence came. Alternatively, if a partial match is found many possibilities arise regarding the “partially matching” gene(s). For example, it may be that the EST comes from a gene belonging to the same family as one or more of the “partially matching” genes. In either case, this provides information that the matching or partially matching gene(s) have already been cloned and sequenced. The cloned gene can then be obtained using known procedures for cloning genes or cDNA. Alternatively, services to clone the gene or cDNA can be retained. If no meaningful matches are obtained, or the partial matches do not satisfy the desired specificity, the researcher is left without a gene sequence containing the EST. In this latter scenario, cloning and sequencing of the gene de novo must occur. It may also be desirable to clone the gene family of the gene from which the EST came. A “gene family” is a set of genes that encode proteins that contain a functional domain for which a consensus sequence can be identified.

After cloned cDNA gene or gene family and/or sequence thereof are obtained, further investigation of gene function can occur. For example, cell lines may be made having various modifications in the endogenous gene to aid in identifying the gene function. Additionally, phenotypic changes may result from the modifications to one or more alleles. The cell lines or a subset thereof can be screened against agents for bioactivity and expression levels of the gene in various tissues may then be used to determine what assays to perform. At each step a functional genomics product is used to make the cell lines, determine expression profiles or determine the appropriate assays to perform.

Clearly, the researcher must spend a significant amount of time and effort obtaining functional genomics products/services, many or most of which the researcher can obtain from a service provider (e.g., clones, clones of gene families, customized DNA libraries, modified cell lines, transgenic animals). The process of obtaining functional genomics products/services requires identifying multiple service providers, ensuring each of the providers has the correct instructions and materials, and managing the logistics between providers (e.g., if results are delayed from one provider, this may cause a significant backup in obtaining results from a second provider). Moreover, the researcher must manage all of the data and products coming from the service providers in a way that makes the data and products useful or informative for future projects.

Accordingly, there is a need in the art for a functional genomics service system that integrates all or virtually all of the desired functional genomics products and services for the researcher, thereby realizing economies of scale. There is a further need in the art for the functional genomics service system to manage the functional genomics data and/or products to better facilitate the use thereof for additional and/or related projects.

### SUMMARY OF THE INVENTION

In one aspect of the invention, a method is provided for integrated genomic services comprising (a) receiving a first request from a customer, wherein said request comprises a first nucleic acid sequence, and an order for at least two genomics products or services; and (b) utilizing said nucleic acid sequence to provide said at least two genomics services or products.

In a further aspect of the invention, a method for providing integrated genomics services comprising (a) receiving a first request from a customer comprising a first nucleic acid sequence and an order for at least one first genomic product or service; (b) receiving a second request from the same or different customer comprising a second nucleic acid sequence and an order for at least one second genomic product or service; and (c) utilizing said first and said second nucleic acid sequences to provide said first and said second genomic product or service to said customers. In each of these aspects of the invention, a recombinase mediated process is preferably used to make the genomic product.

In addition, the invention provides a method for providing an integrated genomic service comprising (a) receiving a first request from a customer comprising a first nucleic acid sequence and an order for at least one genomic product or service; and (b) utilizing said first nucleic acid sequence in a recombinase mediated process to form said at least one genomic product.

### BRIEF DESCRIPTION OF THE DRAWINGS

FIG. 1 is a block diagram of a system for providing integrated genomic services and/or products in accordance with an embodiment of the present invention;

FIG. 2 is a block diagram of a request database used in an embodiment of the present invention;

FIG. 3 is a block diagram of a report database used in an embodiment of the present invention;

FIG. 4 is a block diagram of a genomic services database used in an embodiment of the present invention; and

FIG. 5 is a flow chart showing the procedure for receiving and processing an order for one or more genomics services or products in accordance with an embodiment of the present invention.

### DETAILED DESCRIPTION OF THE PREFERRED EMBODIMENTS

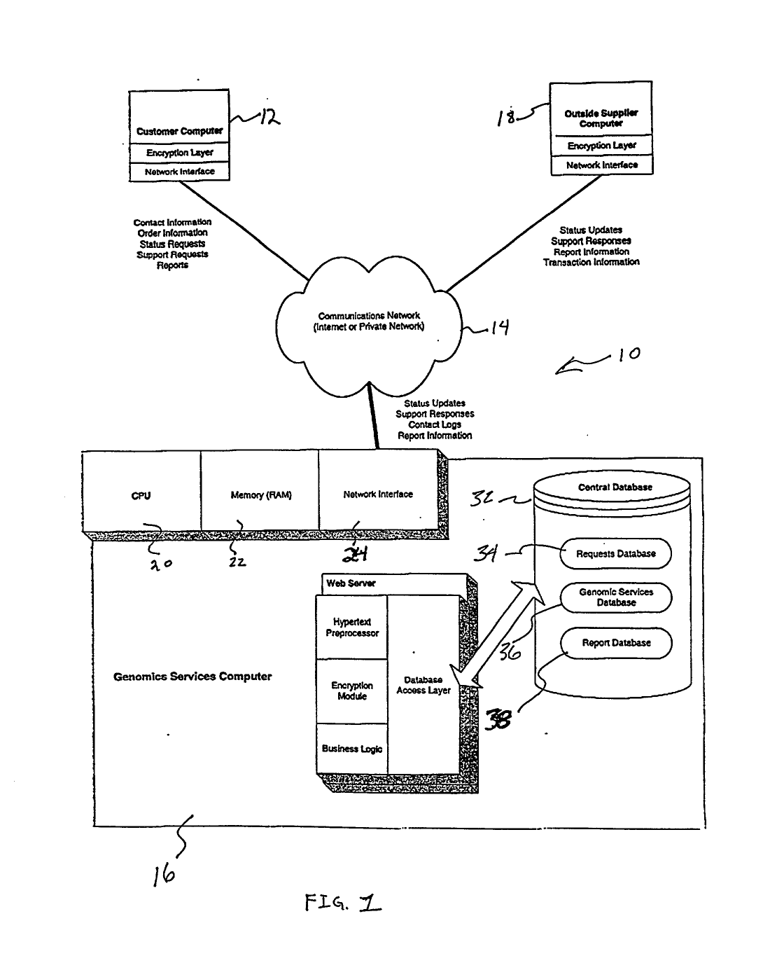

Referring to FIG. 1, system 10 in accordance with an embodiment of the present invention is provided for integrating functional genomic services. System 10 includes customer computer 12, communications network 14, genomic services computer 16, and optionally outside supplier computer 18. Genomics services computer 16 is preferably under the roof of the company (hereinafter “DirectGenomics”) providing the requested integrated genomics services and resulting genomics products. “Genomics services” as used herein means processes used to generate genomics products, for example and without limitation, gene cloning, customized DNA or polypeptide library production, gene expression, custom antibody libraries, transgenic animal production, amino and nucleic acid sequencing etc. “Genomics product” as used herein means a physical product made as a result of performing genomics services. For example, and without limitation, a genomics product is a cloned nucleic acids-cDNA, a cloned gene, a cell line transfected with a cloned cDNA, gene or gene fragment, one or more cell lines with targeted modification(s) to an endogenous nucleic acid a gene, a library of proteins expressed by a plurality of endogenous nucleic acids each with at least one targeted modification, DNA primers, synthesized gene(s), custom DNA libraries, transgenic animals having a targeted modification to an endogenous nucleic acid or a knock-out of one or more endogenous alleles, the phenotyp of modified cells or animals, database of genomic data, databases that correlate genotypic and phenotypic data, and biopharmaceuticals.

Generally, a customer uses customer computer 12, or other suitable communication device (such as a phone or facsimile; although a computer is preferred) to transmit a request over communications network 14XX to genomic services computer 16. Communications network 14 is preferably the internet, an extranet or a combination of the two. It is understood that communications network 14 includes a public switched telephone network, satellite network or any other means for permitting the customer to transmit the request to genomics services computer 16.

Genomics services computer 16 includes:

CPU 20

memory 22; and

network interface 24, which connects genomics services computer 16 to communications network 14.

Memory 22 includes

operating system 26 (such as Windows NT™, MacOS™, or Linux™ for example)

request receiving application 28, which represents a software module having instructions for receiving, processing and saving requests for genomic services;

report receiving application 30, which represents a software module having instructions for receiving, processing and saving data generated from the completed genomics services submitted in the request; and

databases 32.

Referring to FIG. 1 databases 32 include:

requests database 34;

genomic services database 36; and

reports database 38.

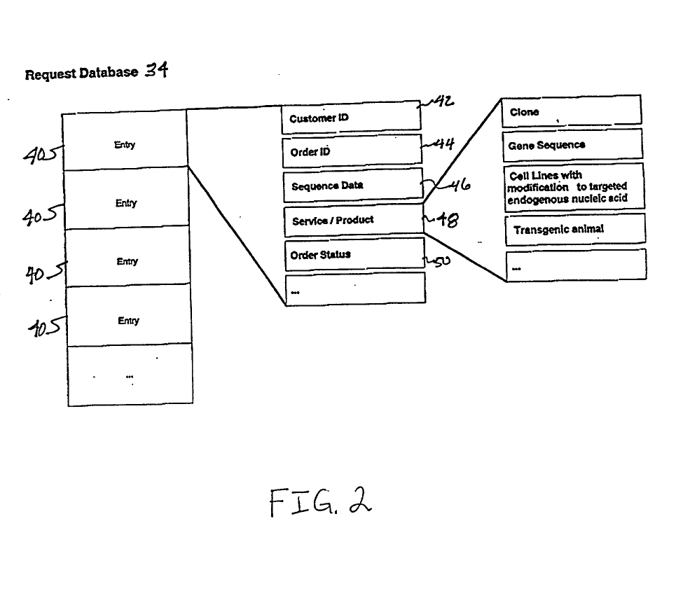

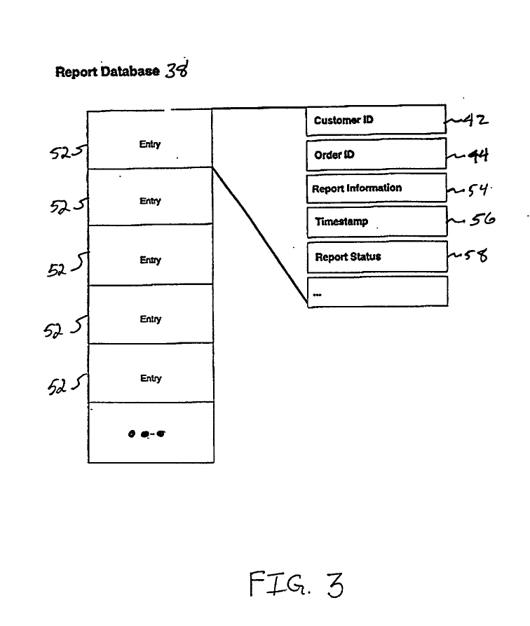

Referring to FIG. 2, requests database 34 contains searchable entries, preferably with hierarchical access schemes to limit access to a particular customer's outstanding request(s) to the customer, and to selected individuals at DirectGenomics. Entries 40 of requests database 34 may include, without limitation, unique customer number 42, unique order identification number 44, sequence data 46 submitted with request, genomics product(s) and/or services ordered 48 with request, and status 50 of each genomics product(s) ordered. Referring to FIG. 3, reports database 38 also contains searchable entries with access limited to the customer providing the customer access to all previous orders and the reports therefore. Entries 52 of reports database may include, without limitation, unique customer number 42, unique order identification number 44, report 54 for each requested genomics product or service, time stamp 56, and report status 56. Report 54 includes data generated and reported from the requested genomics product, for example and without limitation, the nucleic acid sequence of a cloned gene, a protein sequence expressed by the gene, or the results of drug screens agains cell lines expressing the cloned gene etc.

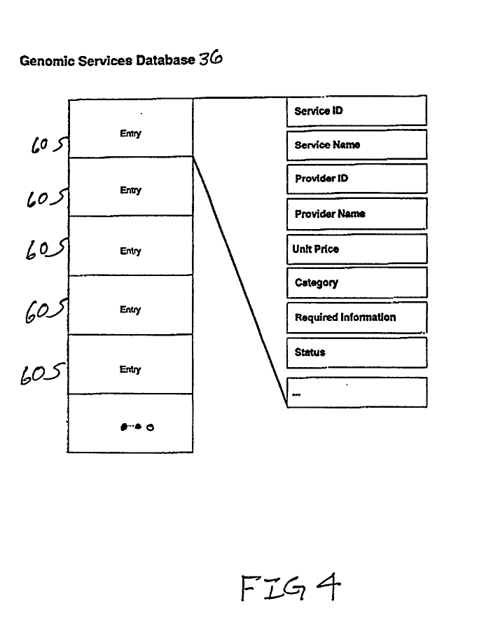

Referring to FIG. 4, genomic services database 36 includes searchable entries 60 to identify companies that provide a given service. Each entry may include the information provided in FIG. 4. Additionally, preferably each company is ranked based on the effectiveness with which it provides a given service. The rank may be based upon such criteria as timeliness, accuracy, expertise, or price. The rank proivdes one means by which the expertise of DirctGenomics may be utilized in identifying from which company to obtain the genomics product or service, if DirectGenomics does not provide it. Preferably DirectGenomics will provide the requested product or service, and would thus select itself. If DirectGenomics does not provide one or more of the services or products, the company rankings are used to select a provider. Alternatively, DirectGenomics may contract for specific products or services not provided by DirectGenomics from one company, such that the specific product or service, if requested, would always be provided by the one company. This provides the ability to obtain bulk discounts as well as access specialized expertise for that one specific product or service. The skilled artisan will recognize that other combinations may be used without exceeding the scope of the present invention.

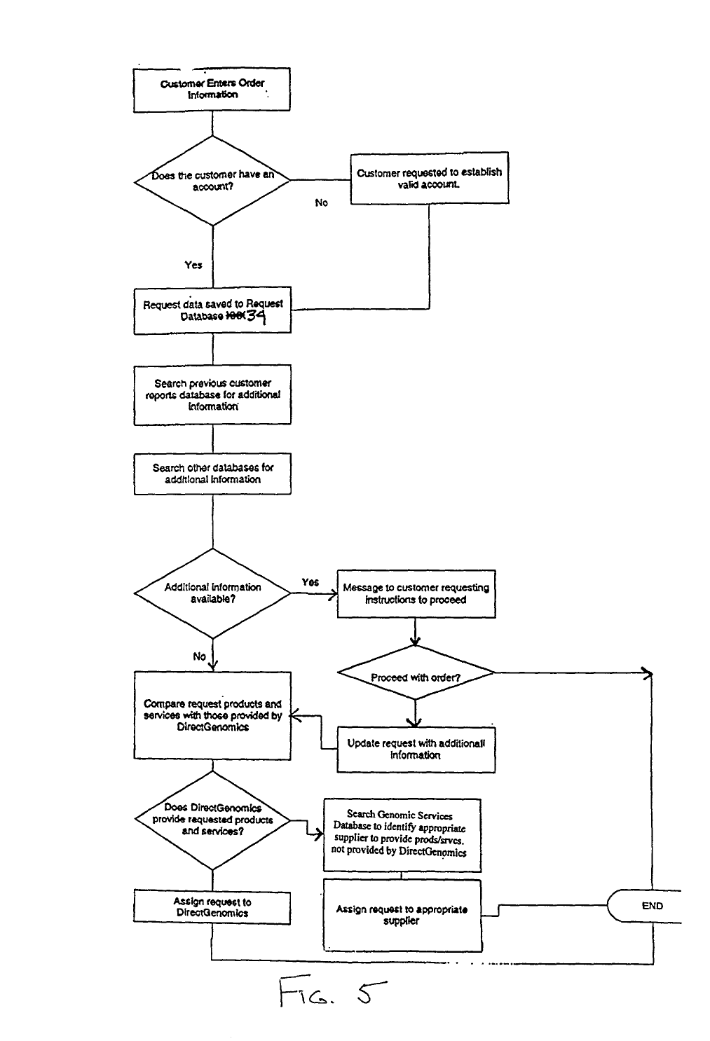

Referring to FIG. 5, request receiving application 28 begins with the customer accessing DirectGenomics website <http://www.directgenomics.com> and accessing customer order page. Customer order page contains general instructions on how to place an order for genomics products and/or services, fields and menus are provided for entering data and selecting criteria (e.g., servies and/or products) necessary for completing the order. For example, and without limitation, the following self-explanatory fields, buttons and menus are provided: customer identification number field and/or cookie therefor, nucleic acid sequence field and/or a pointer to a nucleic acid sequence (e.g., a reference number in a database containing the sequence), pull down menu for selecting one or more (preferably at least two) genomics products, and comments field. After completing order page, the customer clicks the submit button which transmits the request to genomic services computer 16.

For the purposes of this explanation and not by way of any limitation, a list of genomic products requested sent by a hypothetical customer includes (1) a cloned nucleic acid (e.g., cDNA, partial or complete gene), (2) single or multiple cell lines each having a different targeted modification in one or more endogenous genes, (3)single or multiple cell lines having the insertion, substitution or deletion of one or more exogenous genes or modified genes, (4) transgenic animals each having cells which have a targeted modification of one or more endogenous allelles including disruption of gene function (knock-out) or modification of the gene product or its expression level as well as transgenic animals having exogenous nucleic acids incorporated into one or more cells. It is understood that more, less and/or different genomics products from the above can be included in the request. “Genomic services” includes, but is not limited to, phenotyping any of the above cells and/or animals as well as conducting high throughput screening of said nucleic acids, proteins encoded thereby as well as cells and animals containing such nucleic acids.

In processing the request, request receiving application 28, checks the customer identification number 42 to verify that the customer is in good standing (e.g., is registered and credit worthy). If either the check fails, the customer is requested to establish a valid account. After customer verification, the program saves all of the request data into request database 34, and each genomics product requested is initialized with a status of incomplete, and optionally an estimated date for providing the genomics product. Optionally a confirmation of the order is sent back to the customer. Access to a customer request is only given to the customer that submitted the request, and that customer may search the request database at anytime to monitor the status of the request. Preferably, any contact wherein data is sent or viewed over a public network, is done using encrypted connections, or over a private or semi-private secured transmission line.

Next, the nucleic acid sequence, in this example an EST, is used to search the customers completed reports within the system, which are more fully described below, to determine if any related or redundant genomic products had been previously requested and produce. Additionally, request receiving application 28 can search the customer's proprietary databases through a secured link to make the same determination. If there is a redundancy or similarity between work previously completed and the requested genomics products and/or the submitted EST then the program sends off a prescripted message to the customer to provide this additional information. Alternatively or in combination with the electronic message, an individual at DirectGenomics would be notified to contact the customer directly to discuss the additional information. If no redundancy exits (i.e., no additional information is found), the EST sequence is used to search public and/or proprietary databases to determine if the one or more genes comprising the EST or something close thereto had been previously cloned. If so, the program sends off a prescripted message to the customer to provide this additional information.

In either situation, if additional information is available a reply is requested directing DirectGenomics on whether to proceed with the order in light of the additional information. If DirectGenomics is to proceed, the additional information is used to update the request data in request database. In some situations the information may render moot one or more of the requests for a genomic product. For example, if the customer had already obtained a cloned gene containing the submitted EST, and that clone was available, there would be no need to clone it again. Alternatively, if only the sequence is provided and a clone is needed then cloning would be performed.

If no redundant or additional information is identified, or after the request data is updated to reflect the redundant or additional data, request receiving application 28 compares the requested genomics products and services against the products and services provided directly by DirectGenomics. If DirectGenomics does not, or is unable to produce one or more of the genomics products or services, then request receiving application 28 checks genomic services database 36 to determine which supplier can provide the genomics product(s) or service(s) not provided by DirectGenomics. Request receiving application 28 then sends a request for the needed genomics products and services with all other appropriate information to the identified suppliers. In this manner the customer can rely upon DirectGenomics' expertise to either directly provide the requested genomics products and services, or to efficiently obtain the requested product or service in the case when DirectGenomics does not or cannot do so.

DirectGenomics, and the other supplier if any, then begin producing the requested product(s). In the given example, again for the illustrative purposes and without limitation, no additional or redundant data is found, and DirectGenomics is able to provide all of the requested genomics products.

DirectGenomics preferably uses recombinase mediated processes to provide the requested genomic products and services. “Recombinase mediated processes” as used herein is a process that uses a recombinase to enhance the interaction of single- or double-stranded targeting polynucleotide with a single- or double-stranded target nucleic acid. Examples of recombinase mediated processes include, without limitation, the use of recombinase coated single- or double-stranded targeting polynucleotides to form single- or double-D loops with homologous target nucleic acid sequences to either isolate the homologous nucleic acid or to facilitate enhanced homologous reombination (EHR) in vitro or in vivo. It will be recognized that any number of other known molecular biology techniques may be used to produce the genomics products and/or provide the genomics services in accordance with the present invention. In an alternative embodiment at least two genomics products/services are requested. In some embodiments at least one genomic product is provided using a recombinase mediated processes. In a preferred embodiment all requested genomic products and services are provided using a recombinase mediated process, when such process would be logically applicable.

In the present invention, recombinase or Rec-A like recombinase refers to a family of recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to substantially complementary endogenous sequences, or exogenous sequences within a nucleic acid library. The best characterized RecA protein is from RecA, in addition to the wild-type protein a number of mutant RecA proteins have been identified (e.g., RecA803; see Madiraju et al., PNAS USA 85(18):6592 (1988); Madiraju et al, Biochem. 31:10529 (1992); Layery et al., J. Biol. Chem. 267:20648 (1992)). Further, many organisms have RecA-like recombinases with strand-transfer activities (e.g., Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. (ISA) 85: 3683; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimeic, (1984) Cold Spring Harbor Symp. 48: 675; Kmeic, (1986) Cell 44: 545; Kolodner et al., (1987) Proc. Natl. Acad. Sci. USA 84: 5560; Sugino et al., (1985) Proc. Natl. Acad. Sci. USA 85: 3683; Halbrook et al., (1989) J. Biol. Chem. 264: 21403; Eisen et al., (1988) Proc. Natl. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference). Examples of such recombinase proteins include, for example but not limited to: RecA, RecA803, uvsX, and other RecA mutants and RecA-like recombinases (Roca, A. I. (1990) Crit. Rev. Biochem. Molec. Biol. 25: 415), sep1 (Kolodner et al. (1987) Proc. Natl. Acad. Sci. (U.S.A.) 84:5560; Tishkoff et al. Molec. Cell. Biol. 11:2593), RuvC (Dunderdale et al. (1991) Nature 354: 506), DST2, KEM1, XRN1 (Dykstra et al. (1991) Molec. Cell. Biol. 11:2583), STPα/DST1 (Clark et al. (1991) Molec. Cell. Biol. 11:2576), HPP-1 (Moore et al. (1991) Proc. Natl. Acad. Sci. (U.S.A.) 88:9067), other target recombinases (Bishop et al. (1992) Cell 69: 439; Shinohara et al. (1992) Cell 69: 457); incorporated herein by reference. In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli RecA803 mutant with enhanced recombinase activity. Recombinase also includes both yeast and mammalian Rad51 proteins, which form nucleoprotein filaments on single-stranded DNA, and mediate homologous pairing and strand-exchange reactions between ssDNA and homologous double-stranded DNA (Baumann, P., et al., Cell 87:757-766 (1996); Gupta, R. C., et al., Proc. Natl. Acad. Sci. USA 94:463-468 (1997); Sung, P. Science 265:241-1243 (1994); Sung, P. and D. L. Robberson Cell 82: 453-461 (1995), all incorporated herein by reference).

“Targeting polynucleotides”, and grammatical equivalents thereof as used herein are single- or double-stranded, preferably single stranded, polynucleotides. A targeting polynucleotide as used herein may be coated with a RecA-like recombinase depending on the context in which the targeting polynucleotide is used, as will be appreciated by the skilled artisan. A “nucleoprotein filament”, “DNA probe”, or “coated targeting polynucleotide” as used herein are targeting polynucleotides coated with a RecA-like recombinase. Targeting polynucleotides are most preferably two substantially complementary single-stranded polynucleotides. Targeting polynucleotides are generally at least about 5 to 2000 nucleotides long, preferably about 12 to 200 nucleotides long, at least about 200 to 500 nucleotides long, more preferably at least about 500 to 2000 nucleotides long, or longer.

Targeting polynucleotides have at least one sequence, referred to herein as a homology clamp, that substantially corresponds to, or substantially complements at least a portion of a target nucleic acid. The target nucleic acid may be, for example and without limitation, a predetermined endogenous DNA sequence or a to be identified/cloned nucleic acid in a library. A “homology clamp” can specifically hybridize to at least a portion of the target sequence. “Specific hybridization” is defined herein as the formation of hybrids between a targeting polynucleotide (e.g., a polynucleotide of the invention which may include substitutions, deletion, and/or additions as compared to the predetermined target nucleic acid sequence) and a target nucleic acid, wherein the targeting polynucleotide preferentially hybridizes to the target nucleic acid such that, for example, at least one discrete band can be identified on a Southern blot of nucleic acid prepared from target cells that contain the target nucleic acid sequence. It is evident that optimal hybridization conditions will vary depending upon the sequence composition and length(s) of the targeting polynucleotide(s) and target(s), and the experimental method selected by the practitioner. Various guidelines may be used to select appropriate hybridization conditions (see, Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.), which are incorporated herein by reference. As more fully described below, homology clamps serve as templates for targeted homologous pairing with a target nucleic acid.

Thus, for illustrative purposes and without limitation, DirectGenomics would proceed with providing the requested genomics product or services as follows.

1. a recombinase mediated process and the EST are used to produce a clone of at least one gene comprising the EST nucleotide sequence or a sequence substantially corresponding thereto.

2. a report is generated and transmitted to genomic services computer 16, and report receiving application 30 stores this information in reports database 38, updates requests database 34 to change the status of the clone request to complete, and transmits an email to the customer with a prescripted message that the clone has been obtained;

3. the cloned gene is sequenced using known sequencing techniques;

4. number 2 is repeated reporting the results from step 3;

5. a recombinase mediated process, and a plurality of single-stranded targeting polynucleotide sequences (preferably derived from the EST and/or the cloned gene), are used to generate cell lines each having a different targeted modification in the cloned gene;

6. number 2 is repeated reporting the results from step 5;

7. the cell lines are screened for genotypic changes, phenotypic changes, phenotyping small molecule compounds; screening for pharmaceutical drug regulators; screening for biovalidation of drugs, tumorgenicity;

8. number 2 is repeated reporting the results from step 7;

9. a recombinase mediated process, and at least one single-stranded targeting polynucleotide are used to generate transgenic mice having targetedmodified endogenous nucleic acid; and

10. number 2 is repeated reporting the results from step 9.

The skilled artisan will recognize that any of the products generated may be shipped to the customer at any time, or may be stored at DirectGenomics for later use. Additionally, following the completion of the request by DirectGenomics request receiving application 28 closes out the request and removes it from request database 34. However, report receiving application 30 created a redundant file in reports database 38, which is saved for the customer's future use.

Recombinase Mediated Gene Cloning

Gene cloning using recombinase mediated processes comprises the rapid isolation of clones from a DNA library by taking advantage of a recombinase protein, which promotes formation of stable multi-stranded hybrids between targeting polynucleotides (preferably single-stranded) and homologous double-stranded DNA molecules. The targeting of recombinase coated single-stranded probes to homologous sequences at any position in a duplex DNA molecule promotes stable D-loop or double D-loop hybrids, which can be pulled out, cloned and sequenced. The stability of these deproteinized multi-stranded hybrid molecules at any position in duplex molecules allows the application of D-loop methods to many different dsDNA substrates, including duplex DNA from cDNA, genomic DNA, or YAC, BAC or PAC libraries.

In a preferred embodiment, the targeting polynucleotides are attached to a separation moiety that has a binding partner attached to a solid support, such as antibodies (when antigens are used), streptavidin (when biotin is used), or as chemically derivatized particles, plates affinity matrix, non polar surface, ligand receptor, etc. In a preferred embodiment, partial cDNA or EST-size fragments, prepared as biotinylated-ssDNA probes, are used to probe cDNA libraries for the formation of stable biotinylated-probe:target hybrids. Thus, in one embodiment a biotinylated single-stranded targeting polynucleotide (e.g. an EST), preferably its substantial complement, and a recombinase are contacted with a nucleic acid library (e.g., cDNA library, genomic DNA library, YAC library, BAC library or PAC library, mammalian library, mouse library, mixed species library, functional library (i.e., where each member codes for a functional protein)), or complex nucleic acid mixtures such as, without limitation, genomic DNA. The probe:target hybrids are selectively captured on streptavidin-coated magnetic beads. The enriched plasmid population is eluted from the beads, precipitated, resuspended, and used to transform bacteria or the cells. The resulting colonies are screened by PCR and colony hybridization to identify the desired clones. Using this method over 100,000 fold enrichment of the desired clones can be achieved. “Cloning” as used herein means the isolation and amplification of a target sequence.

Other libraries may include libraries made from any number of different target cells as is known in the art. By “target cells” herein is meant prokaryotic or eukaryotic cells. Suitable prokaryotic cells include, but are not limited to, bacteria such as E. coli, Bacillus species, and extremophile bacteria such as thermophiles, etc. Preferably, the prokaryotic target cells are recombination competent. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, rice, potato, alfalfa, sunflower, etc.; and animal cells, including fish, avian and mammalian cells. Suitable fish cells include, but are not limited to, those from species of salmon, trout, tilapia, tuna, carp, flounder, halibut, swordfish, cod and zebra fish. Suitable avian cells include, but are not limited to, those of chicken, duck, quail, pheasant and turkey, and other jungle foul or game birds. Suitable mammalian cells include, but are not limited to, cells from horse, cow, buffalo, deer, sheep, rabbit, rodents such as mouse, rat, hamster and guinea pig, goat, pig, primates, marine mammals including dolphins and whales, as well as cell lines, such as human cell lines of any tissue or stem cell type, and stem cells, including pluripotent and non-pluripotent, and non-human zygotes. In some embodiments, preferred cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In a preferred embodiment, after isolation, the target nucleic acids are cloned and sequenced, as is known in the art. As will be appreciated by those in the art, when a target gene is isolated, it may be that the isolated target sequence is not the full length gene: that is, it does not contain a full open reading frame. In this case, either the experiments can be run again, using either the same targeting polynucleotides or targeting polynucleotides based on some of the new sequence. In addition, multiple experiments may be run to enrich for the desired target sequence. For instance, multiple 5′ and 3′ derived probes can be used in succession to obtain full length gene clones.

Additionally, the process may be used to identify of functional domains, and validate the selected sequences. The high-throughput automated analysis of the gene clones (cDNAs, genomic DNA, alternative splice forms, polymorphisms, gene family members) will provide informative analysis of the qualitative differences between expressed genes (gene profiling). Sequence analysis of the isolated cDNAs and genomic DNA allows diagnostic testing for single and multiple nucleotide polymorphisms, loss of heterozygosity (LOH), and other chromosomal abnormalities. Differences in gene families and mRNA spliced isoforms can be elucidated, and information can be provided on the nature of the mRNA. Libraries of clones obtained at the end of the process will mimic the difference between normal and genetic disorders (or between any differential event). These libraries can be used to screen for genetic signatures and the technology can elucidate precise potential domains of therapeutic intervention within coding sequences of the gene, including catalytic domains (ie, kinases, phosphatases, proteases), protein-protein interaction domains, truncated receptors and soluble receptors.

As in all the recombinase mediated processes described herein, it is preferred to use a single-stranded targeting polynucleotide, and more preferable to also use the substantial complement thereof, in combination with a recombinase. Moreover, it is preferred to first coat the targeting polynucleotide with the recombinase prior to contacting the nucleic acid library. In general, as will be appreciated by those in the art, targeting polynucleotides may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence.

Cloning using recombinase mediated processes is further described in the following publications: WO 00/63365, WO 99/60108, WO 00/56872, WO 99/37755, U.S. Pat. Nos. 5,948,653, 6,074,853, 5,763,240, 5,929,043, 5,989,879, and U.S. Ser. No. 09/654,108, all of which are incorporated herein in their entirety by reference.

Recombinase Mediated Targeted Cell Modification

Generally, any predetermined endogenous DNA sequence, such as a gene sequence, can be altered by homologous recombination (which includes gene conversion) with an exogenous targeting polynucleotides (preferably a substantially complementary pair of single-stranded targeting polynucleotides). The targeting polynucleotides have at least one homology clamp, which substantially corresponds to or is substantially complementary to at least a portion of the targeted endogenous DNA sequence (such as a cloned gene). The targeting polynucleotides are introduced into the cell with a RecA-like recombinase (e.g., RecA). Typically, a targeting polynucleotide (or complementary polynucleotide pair) has a portion or region having a sequence that is not present in the targeted endogenous sequence (i.e., a nonhomologous portion or mismatch) which may be as small as a single mismatched nucleotide, several mismatches, or may span up to about several kilobases or more of nonhomologous sequence. Generally, such nonhomologous portions are flanked on each side by homology clamps, although a single flanking homology clamp may be used. Nonhomologous portions are used to make insertions, deletions, and/or replacements in a predetermined endogenous targeted DNA sequence, and/or to make single or multiple nucleotide substitutions in a predetermined endogenous target DNA sequence so that the resultant recombined sequence (i.e., a targeted recombinant endogenous sequence) incorporates some or all of the sequence information of the nonhomologous portion of the targeting polynucleotide(s). Thus, the nonhomologous regions are used to make variant sequences, i.e. targeted sequence modifications. Additions and deletions may be as small as 1 nucleotide or may range up to about 2 to 4 kilobases or more. In this way, site directed modifications may be done in a variety of systems for a variety of purposes.

The targeting polynucleotides are derived from a known endogenous target sequence, a cloned gene for example. In a preferred embodiment, a plurality of targeting polynucleotides are designed, such that upon targeted homologous recombination with the target sequence a plurality of targeted modification is introduced into the targeted endogenous sequence of a plurality of cells. By using a plurality of targeting polynucleotides, each designed to introduce a different modification to the targeted endogenous nucleic acid, a plurality of cell lines is made each having a different modification in the targeted endogenous nucleic acid sequence.

Typically, a targeting polynucleotide of the invention is coated with at least one recombinase and is conjugated to a cell-uptake component, and the resulting cell targeting complex is contacted with a target cell under uptake conditions (e.g., physiological conditions) so that the targeting polynucleotide and the recombinase(s) are internalized in the target cell. A targeting polynucleotide may be contacted simultaneously or sequentially with a cell-uptake component and also with a recombinase; preferably the targeting polynucleotide is contacted first with a recombinase, or with a mixture comprising both a cell-uptake component and a recombinase under conditions whereby, on average, at least about one molecule of recombinase is noncovalently attached per targeting polynucleotide molecule and at least about one cell-uptake component also is noncovalently attached. Most preferably, coating of both recombinase and cell-uptake component saturates essentially all of the available binding sites on the targeting polynucleotide. A targeting polynucleotide may be preferentially coated with a cell-uptake component so that the resultant targeting complex comprises, on a molar basis, more cell-uptake component than recombinase(s). Alternatively, a targeting polynucleotide may be preferentially coated with recombinase(s) so that the resultant targeting complex comprises, on a molar basis, more recombinase(s) than cell-uptake component.

The two complementary single-stranded targeting polynucleotides are simultaneously or contemporaneously introduced into a target cell harboring a predetermined endogenous target sequence, with a RecA-like recombinase protein. Under most circumstances, it is preferred that the targeting polynucleotides are incubated with RecA or other recombinase prior to introduction into a target cell, so that the recombinase protein(s) may be “loaded” onto the targeting polynucleotide(s), to coat the nucleic acid. Incubation conditions for such recombinase loading are described infra, and also in U.S. Pat. Nos. 5,670,316, 5,273,881, 5,223,414, each of which is incorporated herein by reference. A targeting polynucleotide may contain a sequence that enhances the loading process of a recombinase, for example a RecA loading sequence is the recombinogenic nucleation sequence poly[d(A-C)], and its complement, poly[d(G-T)]. The duplex sequence poly[d(A-C).d(G-T)n, where n is from 5 to 25, is a middle repetitive element in target DNA.

Once variant target sequences are made, any number of different phenotypic screens may be done. As will be appreciated by those in the art, the type of phenotypic screening will depend on the mutant target nucleic acid and the desired phenotype; a wide variety of phenotypic screens are known in the art, and include, but are not limited to, phenotypic assays that measure alterations in multicolor fluorescence assays; cell growth and division (mitosis: cytokinesis, chromosome segregation, etc); cell proliferation; DNA damage and repair; protein-protein interactions, include interactions with DNA binding proteins; transcription; translation; cell motility; cell migration; cytoskeletal (microtubule, actin, etc) disruption/localization; intracellular organelle, macromolecule, or protein assays; receptor internalization; receptor-ligand interactions; cell signaling; neuron viability; endocytic trafficking; cell/nuclear morphology; activation of lipogenesis; gene expression; cell-based and animal-based efficacy and toxicity assays; apoptosis; cell differentiation; radiation resistance/sensitivity; chemical resistance/sensitivity; permeability of drugs; pharmocokinetics; pharmacodynamics; pharmacogenomics in cells and animals; nucleus-to-cytoplasm translocation; inflammation-inflammatory tissue injury; wound healing; cell ruffling; cell adhesion; drug induced redistribution of target protein; immunoassays for diagnostics and the emerging field of proteomics.; cell sorting; phenotypic screening of cells and animals; phenotyping small molecule drug inhibitors; biovalidation of drug targets in transgenic recombinant cell and animal phenotypes; single and multiple nucleotide polymorphisms diagnostics; loss of heterozygosity (loh) and other chromosomal aberration diagnostics; in situ gene targeting (hybridization) in cells, tissues, and animals; in situ gene recombination in cells and animals; and gene delivery and therapy. See Keller, Current Opin. In Cell Biol. 7:862 (1995); Hsin et al., Nature 399(6743):362 (1999); Giuliano et al., Tibtech 16:135 (1998); Conway et al., J. Biomolecular Screening 4:75 (1999); Giulano et al., J. Biomolecular Screening 2:249 (1997); Forrester et al., Genetics 148:151 (1998); Reiter et al., Genes Dev. 13:2983 (1999); Carmeliet et al., Nature 380:435 (1996); Ferrara et al, Nature 380:439 (1996); Hidaka et al., Genetics 96:7370 (1999); DeWeese et al., Medical Sci. 95:11915 (1998); Aszterbaum et al., Nature Med. 5:1285 (1999); Abuin et al., Mol. Cell. Biol. 20:149 (2000); de Wind et al., Nature Genetics 23:359 (1999); Gailani et al., Nature Genet. 14:78 (1996); Tanzi et al., Neurobiol. Dis. 3:159 (1996); Jensen et al., Artherosclerosis 120:57 (1996); Lipkin et al., Nature Genetics 24:27 (2000); Chen et al., Genes Dev. 11:2958 (1997) and Brown et al., Genes Dev. 11:2972 (1997); and and U.S. Pat. Nos. 5,989,835 and 6,027,877.

Recombinase mediated targeted cell modification processes are further described in the following publications WO 00/63365, WO 99/60108, WO 00/56872, WO 99/37755, U.S. Pat. Nos. 5,948,653, 6,074,853, 5,763,240, 5,929,043, 5,989,879, and U.S. Ser. No. 09/654,108, all of which are incorporated herein in their entirety by reference.

Recombinase Mediated Targeted Transgenic Animal Production

Exogenous targeting polynucleotides can be used to inactivate, decrease or alter the biological activity of one or more genes in a cell (or transgenic nonhuman animal or plant). This finds particular use in the generation of animal models of disease states, or in the elucidation of gene function and activity, similar to “knock out” experiments. Alternatively, the biological activity of the wild-type gene may be either decreased, or the wild-type activity altered to mimic disease states. This includes genetic manipulation of non-coding gene sequences that affect the transcription of genes, including, promoters, repressors, enhancers and transcriptional activating sequences.

In a preferred embodiment, eukaryotic cells are used. For making transgenic nonhuman animals (which include homologously targeted non-human animals) embryonal stem cells (ES cells) and fertilized zygotes are preferred. In a preferred embodiment, embryonal stem cells are used. Murine ES cells, such as AB-1 line grown on mitotically inactive SNL76/7 cell feeder layers (McMahon and Bradley, Cell 62: 1073-1085 (1990)) essentially as described (Robertson, E. J. (1987) in Teratocarcinomas and Embrvonic Stem Cells: A Practical Approach. E. J. Robertson, ed. (oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 21-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germline transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In a preferred embodiment, non-human zygotes are used, for example to make transgenic animals, using techniques known in the art (see U.S. Pat. No. 4,873,191). Preferred zygotes include, but are not limited to, animal zygotes, including fish, avian and mammalian zygotes. Suitable fish zygotes include, but are not limited to, those from species of salmon, trout, tuna, carp, flounder, halibut, swordfish, cod, tulapia and zebrafish. Suitable bird zygotes include, but are not limited to, those of chickens, ducks, quail, pheasant, turkeys, and other jungle fowl and game birds. Suitable mammalian zygotes include, but are not limited to, cells from horses, cattle, buffalo, deer, sheep, rabbits, rodents such as mice, rats, hamsters and guinea pigs, goats, pigs, primates, and marine mammals including dolphins and whales. See Hogan et al., Manipulating the Mouse Embryo (A Laboratory Manual), 2nd Ed. Cold Spring Harbor Press, 1994, incorporated by reference.

In general, transgenic animals are made with any number of changes. Exogeneous sequences, or extra copies of endogeneous sequences, including structural genes and regulatory sequences, may be added to the animal, as outlined below. Endogeneous sequences (again, either genes or regulatory sequences) may be disrupted, i.e. via insertion, deletion or substitution, to prevent expression of endogeneous proteins. Alternatively, endogeneous sequences may be modified to alter their biological function, for example via mutation of the endogeneous sequence by insertion, deletion or substitution.

The methods of the present invention are useful to add exogenous DNA sequences, such as exogenous genes or regulatory sequences, extra copies of endogenous genes or regulatory sequences, or exogeneous genes or regulatory sequences, to a transgenic plant or animal. This may be done for a number of reasons: for example, adding one or more copies of a wild-type gene can increase the production of a desirable gene product; adding or deleting one or more copies of a therapeutic gene can alleviate a disease state, or to create an animal model of disease. Adding one or more copies of a modified wild type gene may be done for the same reasons. Adding therapeutic genes or proteins may yield superior transgenic animals, for example for the production of therapeutic or nutriceutical proteins. Adding human genes to non-human mammals may facilitate production of human proteins and adding regulatory sequences derived from human or non-human mammals may be useful to increase or decrease the expression of endogenous or exogenous genes. Such inserted genes may be under the control of endogenous or exogenous regulatory sequences, as described herein.

The methods of the invention are also useful to modify endogeneous gene sequences, as outlined below. Suitable endogenous gene targets include, but are not limited to, genes which encode peptides or proteins including enzymes, structural or soluble proteins, as well as endogeneous regulatory sequences including, but not limited to, promoters, transcriptional or translational sequences, repetitive sequences including oligo[d(A-C)ₙ.d(G-T)ₙ], oligo[d(A-T)]ₙ, oligo[d(C-T)]ₙ, etc. Examples of such endogenous gene targets include, but are not limited to, genes which encode lactoglobulins including both a-lactoglobulin and β-lactoglobulin; casein, including both α-casein, β-casein and κ-casein; albumins, including serum albumin, particularly human and bovine; immunoglobulins, including IgE, IgM, IgG and IgD and monoclonal antibodies; globin; integrin; hormones; growth factors, particularly bovine and human growth factors, including transforming growth factor, epidermal growth factor, nerve growth factors, etc.; collagen; interleukins, including IL-1 to IL-17; a major histocompatibility antigen (MHC); G-protein coupled receptors (GPCR); nuclear receptors; ion channels; multidrug resistance genes; amyloid proteins; enzymes, including esterases, proteases (including tissue plasminogen activator (tPA)), lipases, carbohydrases, etc.; APRT, HPRT; leptin; tumor suppressor genes; provirus; prions; OTC; CFTR; sugar transferases such as alpha-galactosyl transferase (galT) or fucosyl transferase; a milk or urine protein gene including the caseins, lactoferrin and whey proteins; oncogenes; cytokines, particularly human; transcription factors; and other pharmaceuticals. Any or all of these may also be suitable exogeneous genes to add to a genome using the methods outlined herein.

The endogenous target gene may be disrupted in a variety of ways. The term “disrupt” as used herein comprises a change in the coding or non-coding sequence of an endogenous nucleic acid that alters the transcription or translation of an endogenous gene. In a preferred embodiment, a disrupted gene will no longer produce a functional gene product. Generally, disruption may occur by either the insertion, deletion or frame shifting of nucleotides.

Recombinase mediated targeted transgenic animal production is further described in the following publications WO 00/63365, WO 99/60108, WO 00/56872, WO 99/37755, U.S. Pat. Nos. 5,948,653, 6,074,853, 5,763,240, 5,929,043, 5,989,879, and U.S. Ser. No. 09/654,108, all of which are incorporated herein in their entirety by reference.

This invention describes integrating many of the functional genomic services resulting in the benefits of economies of scale. Additionally, utilizing recobinase mediated processes further enhances the benefits of the stream lined integrated functional genomics services. More specifically the recombinase mediated processes specifically, efficiently and reliably target and isolate specific DNA molecules for applications such as DNA cloning; biovalidation of drug targets; DNA modification, including mutagenesis, gene shuffling and evolution; isolation of gene families, orthologs, and paralogs; identification of alternatively spliced isoforms; gene mapping; diagnostic testing for single and multiple nucleotide polymorphisms; differential gene expression and genetic profiling; nucleic acid library production, subtraction and normalization; in situ gene targeting (hybribidization) in cells; in situ gene recombination in cells and animals; high throughput phenotype screening of cells and animals; phenotyping small molecule compounds; screening for pharmaceutical drug regulators; and biovalidation of drugs in transgenic recombinant cells and animals.

The foregoing description, for purposes of explanation, used specific nomenclature to provide a thorough understanding of the invention. Nevertheless, the foregoing descriptions of the preferred embodiments of the present invention are presented for purposes of illustration and description and are not intended to be exhaustive or to limit the invention to the precise forms disclosed; obvious modifications and variations are possible in view of the above teachings. Accordingly, it is intended that the scope of the invention be defined by the following claims.

What is claimed is:

1. A method for providing integrated genomic services comprising:
(a) receiving a first request from a customer, wherein said request comprises a first nucleic acid sequence, and an order for at least two genomics products; and
(b) utilizing said nucleic acid sequence to provide said at least two genomics services or products.

2. The method according to claims 1, 11 further comprising:
(c) storing a first genomic product report for each of said at least two genomics products in a customer report database, wherein said first genomic product report contains searchable genomic product data.

3. The method according to claim 2 further comprising:
(d) receiving a second request from said customer; wherein said request comprises a second order for at least one genomics product, and a second nucleic acid sequence;
(e) comparing said second order and/or said second nucleic acid sequence against said genomic product report to determine if said second request or nucleic acid sequence is redundant.

4. The method according to claim 1, 11, 2 or 3, wherein said at least one genomic product is selected from the group consisting of a nucleic acid clone, a genotypically modified cell, a transgenic genotypically modified animal.

5. The method according to claim 4, wherein said genotypically modified cell line comprises a plurality of cell lines, wherein at least two of said cell lines have a different genotypic modification

6. The method according to claim 4, wherein said nucleic acid clone comprises a plurality of clones representing at least a subset of a gene family.

7. The method according to claim 4, wherein said at least one genomic product is made by a recombinase mediated process.

8. The method according to claim 7, wherein said recombinase mediated process is selected from the group consisting of:
(i) cloning a nucleic acid by contacting a nucleic acid library with first and second substantially complementary single-stranded targeting polynucleotides and a recombinase, wherein said first single-stranded targeting polynucleotide comprises said first nucleic acid sequence or a homologue thereof, and isolating said nucleic acid;
(ii) producing a modified cell with a targeted sequence modification by introducing into a cell first and second substantially complementary single-stranded targeting polynucleotides and a recombinase, wherein said first single-stranded targeting polynucleotide comprises said first nucleic acid sequence or a homologue thereof, and further comprises a homology clamp substantially corresponding to or substantially complementary to a pre-selected target DNA sequence, and identifying a cell having said targeted sequence modification; and
(iii) producing a transgenic animal with a modified preselected DNA sequence, by introducing into a zygote first and second substantially complementary single-stranded targeting polynucleotides and a recombinase, wherein said first single-stranded targeting polynucleotide comprises said first nucleic acid sequence or a homologue thereof and further comprises a homology clamp substantially corresponding to or substantially complementary to a pre-selected wild-type target DNA sequence, wherein said pre-selected wild-type target DNA sequence is modified by homologous recombination with at least one of said first or second substantially complementary single-stranded targeting polynucleotides, and generating said transgenic non-human mammal from said zygote.

9. A method for providing integrated genomics services comprising:
(a) receiving a first request from a customer comprising a first nucleic acid sequence and an order for at least one first genomic product or service;
(b) receiving a second request from the same or different customer comprising a second nucleic acid sequence and an order for at least one second genomic product or service; and
(c) utilizing said first and said second nucleic acid sequences to provide said first and said second genomic product or service to said customers.

10. A method for providing an integrated genomic service comprising:
(a) receiving a first request from a customer comprising a first nucleic acid sequence and an order for at least one genomic product or service; and
(b) utilizing said first nucleic acid sequence in a recombinase mediated process to for said at least one genomic product.

11. A computer program for integrating the provision of genomic services and products comprising:
a request receiving module including instructions for:
(a) receiving a first request from a customer, wherein said request comprises a first nucleic acid sequence, and an order for at least two genomics products, and
(b) processing said request to obtain said at least two genomics products.

12. The computer program according to claim 11, wherein said processing step further comprises:
(i) saving said first request in as request database,
(ii) searching databases to determine if said first request or said first nucleic acid is wholly or partially redundant to information within said databases, and
(iii) updating said first request if any additional information is found in step (ii)
